(19) United States
(12) Patent Application Publication
(10) Pub. No.: US 2012/0078113 A1
(43) Pub. Date: Mar. 29, 2012

(54) Convergent parameter instrument

(75) Inventors: Jennifer J. Whitestone, Germantown, OH (US); Steven H. Mersch, Germantown, OH (US)

(73) Assignee: Point of Contact, LLC

(21) Appl. No.: 12/924,452

(22) Filed: Sep. 28, 2010

(51) Int. Cl. A61B 6/00

(52) U.S. Cl. 600/474

(57) ABSTRACT

The present invention relates to a convergent parameter instrument and method for performing real-time imaging using multiple imaging techniques. More particularly, the present invention relates to a handheld convergent parameter instrument providing some or preferably all of real-time imaging, including surface mapping, color imaging, perfusion imaging, thermal imaging, and near infrared spectroscopy, and including a common control set and a common display.

### BACKGROUND OF THE INVENTION

(a) Field of the Invention

The present invention relates to a convergent parameter instrument and method for performing real-time imaging using multiple imaging techniques. More particularly, the present invention relates to a handheld convergent parameter instrument providing some or preferably all of real-time imaging, including surface mapping, color imaging, perfusion imaging, thermal imaging, and near infrared spectroscopy, and including a common control set and a common display.

(b) Background of the Invention

Skin, the largest organ of the body, has been essentially ignored in medical imaging. No standard of care regarding skin imaging exists. Computerized Tomography (“CT”), Magnetic Resonance Imaging (“MRI”), and ultrasound are routinely used to image within the body for signs of disease and injury. Researchers and commercial developers continue to advance these imaging technologies to produce improved pictures of internal organs and bony structures. Clinical use of these technologies to diagnose and monitor subsurface tissues is now a standard of care. However, no comparable standard of care exists for imaging skin. Skin assessment has historically relied on visual inspection augmented with digital photographs. Such an assessment does not take advantage of the remarkable advances in nontraditional surface imaging, and lacks the ability to quantify the skin's condition, restricting the clinician's ability to diagnose and monitor skin-related ailments. Electronically and quantitatively recording the skin's condition with different surface imaging techniques will aid in staging skin-related illnesses that affect a number of medical disciplines such as plastic surgery, wound healing, dermatology, endocrinology, oncology, and trauma.

Pressure ulcers are a skin condition with severe patient repercussions and enormous facility costs. Pressure ulcers cost medical establishments in the United States billions of dollars annually. Patients who develop pressure ulcers while hospitalized often increase their length of stay to 2 to 5 times the average. The pressure ulcer, a serious secondary complication for patients with impaired mobility and sensation, develops when a patient stays in one position for too long without shifting their weight. Constant pressure reduces blood flow to the skin, compromising the tissue. A pressure ulcer can develop quickly after a surgery, often starting as a reddened area, but progressing to an open sore and ultimately, a crater in the skin.

Other skin injuries include trauma and burns. Management of patients with severe burns and other trauma is affected by the location, depth, and size of the areas burned, and also affects prediction of mortality, need for isolation, monitoring of clinical performance, comparison of treatments, clinical coding, insurance billing, and medicolegal issues. Current measurement techniques, however, are crude visual estimates for burn location, depth, and size. Depth of the burn in the case of an indeterminate burn is often a “wait and see” approach. Accurate initial determination of burn depth is difficult even for the experienced observer and nearly impossible for the occasional observer. Total Burn Surface Area (“TBSA”) measurements require human input of burn location, severity, extent, and arithmetical calculations, with the obvious risk of human error.

An additional skin ailment is vascular malformation (“VM”). VMs are abnormal clusters of blood vessels that occur during fetal development, but are sometimes not visible until weeks or years after birth. Without treatment, the VM will not diminish or disappear but will proliferate and then involute. Treatment is reserved for life or vision-threatening lesions. A hemangioma may appear to present like a VM. However, it is important to distinguish hemangiomas from the vascular malformations in order to recommend interventions such as lasers, interventional radiology, and surgery. One difference between the hemangioma and vascular malformation can be the growth rate as the hemangiomas grow rapidly compared to the child's growth. Other treatments such as compression garments and drug therapy require a quantitative means of determining efficacy. MRI, ultrasonography, and angiograms are used to visualize these malformations, but are costly and sometimes require anesthesia and dye injections for the patient. A need exists with all skin conditions to enable quantification of changes of the anomalies, to prescribe interventions and determine treatment outcomes.

### SUMMARY OF THE INVENTION

The present invention addresses the shortcomings of the prior art and provides a convergent parameter instrument to quantify skin data for rapid and accurate diagnosis and prognosis for a broad range of medical conditions. This convergent parameter instrument is a handheld system which brings together a variety of imaging techniques to digitally record parameters relating to skin condition.

The present invention provides a skin imaging system that is high tech, low-cost, robust, portable, and accessible at the point of care. The instrument integrates some or preferably all of high resolution color imaging, surface mapping, perfusion imaging, thermal imaging, and Near Infrared (“NIR”) spectral imaging. Digital color photography is employed for color evaluation of skin disorders. Use of surface mapping to accurately measure body surface area and reliably identify wound areas has been proven. Perfusion mapping has been employed to evaluate burn wounds and trauma sites. Thermal imaging is an accepted and efficient technique for studying skin temperature as a tool for medical assessment and diagnosis. NIR spectral imaging may be used to measure skin hydration, an indicator of skin health and an important clue for a wide variety of medical conditions such as kidney disease or diabetes. Visualization of images acquired by the different modalities are be controlled through a common control set, such as user-friendly touch screen controls, graphically displayed as 2D and 3D images, separately or integrated, and enhanced using image processing to highlight and extract features. All skin parameter instruments are non-contact which means no additional risk of contamination, infection or discomfort. All scanning modalities may be referenced to the 3D surface acquired by the 3D surface mapping instrument. Combining the technologies creates a convergent parameter system with capability to assess injury to and diseases of the skin.

In one embodiment, the present invention is a convergent parameter instrument comprising: a color imaging module, a surface mapping module, a thermal imaging module, a perfusion imaging module, a near infrared spectroscopy module, a common control set for controlling each of the modules, a common display for displaying images acquired by each of the modules, and a central processing unit for processing image data acquired by each of the modules, the central processing unit in electronic communication with each of the modules, the common control set, and the common display.

In another embodiment, the present invention is a convergent parameter instrument comprising a body incorporating a common display, a common control set, a central processing unit, and between two and four imaging modules selected from the group consisting of: a color imaging module, a surface mapping module, a thermal imaging module, a perfusion imaging module, and a near infrared spectroscopy module. In this embodiment, the central processing unit is in electronic communication with the common display, the common control set, and each of the selected imaging modules, and each of the selected imaging modules are controllable using the common control set, and images acquired by each of the selected imaging modules are viewable on the common display. In this embodiment, the instrument is capable of incorporating at least one additional module from the group into the body, the at least one additional module, once incorporated, being controllable using the common control set and in electronic communication with the central processing unit, and wherein images acquired by the at least one additional module are viewable on the common display.

In a further embodiment, the present invention is a method for quantitatively assessing an imaging subject's skin, comprising: (a.) acquiring at least two skin parameters using a convergent parameter instrument through a combination of at least two imaging techniques, each of the at least two imaging techniques being selected from the group consisting of: (1.) acquiring high resolution color image data using a high resolution color imaging module, (2.) acquiring surface mapping data using a surface mapping module, (3.) acquiring thermal image data using a thermal imaging module, (4.) acquiring perfusion image data using a perfusion imaging module, and (5.) acquiring hydration data using a near infrared spectroscopy module, (b.) using the convergent parameter instrument to select and quantify an imaging subject feature visible in the at least one image, and (c.) assessing the imaging subject based on the quantified imaging subject feature.

In yet another embodiment, the present invention is a method for evaluating a skin malady at a location remote from medical facilities comprising the steps of: (a.) selecting a reference image of the type of malady to be evaluated from a database of reference images, (b.) acquiring at least one image of the skin malady using a handheld convergent parameter instrument, and (c.) evaluating the skin malady by comparing the at least one acquired image with the reference image, wherein the database of reference images is accessed using the handheld convergent parameter instrument.

### BRIEF DESCRIPTION OF THE DRAWINGS

A better understanding of the present invention will be had upon reference to the following description in conjunction with the accompanying drawings, wherein:

FIG. 1A shows a back view of an embodiment of a convergent parameter instrument;

FIG. 1B shows a front view of the embodiment of the convergent parameter instrument;

FIG. 1C shows a perspective view of the embodiment of the convergent parameter instrument; and

FIG. 2 shows a schematic diagram of a convergent parameter instrument.

FIG. 3 is a flowchart of a method for using a convergent parameter instrument.

### DETAILED DESCRIPTION OF THE PREFERRED EMBODIMENT

The present invention involves the integration of up to five imaging modules into a handheld convergent parameter instrument 10. Each imaging module utilizes a common control set 12 and a common display 14.

The first imaging technique is high resolution color digital photography, used for the purpose of medical noninvasive optical diagnostics and monitoring of diseases. Digital photography, when combined with controlled solid state lighting, polarization filtering, and coordinated with appropriate image processing techniques, derives more information that the naked eye can discern. Clinically inspecting visible skin color changes by eye is subject to inter- and intraexaminer variability. The use of computerized image analysis has therefore been introduced in several fields of medicine in which objective and quantitative measurements of visible changes are required. Applications range from follow-up of dermatological lesions to diagnostic aids and clinical classifications of dermatological lesions. For example, computerized color analysis allows repeated noninvasive quantitative measurements of erythema resulting from a local anesthetic used to inhibit edema and improve circulation in burns.

In one embodiment, the present invention includes a color imaging module 16, a state of the art, high definition color imaging array, either a complimentary metal oxide semiconductor (“CMOS”) or charge-coupled device (“CCD”) imaging array. The definition of “high resolution” changes as imaging technology improves, but at this time is interpreted as a resolution of at least 5 megapixels. The inventors anticipate using higher resolution imaging arrays as imaging technology improves. The color image can be realized by the use of a Bayer color filter incorporated with the imaging array. In a preferred embodiment, the color image is realized by using sequential red, green, and blue illumination and a black and white imaging array. This preferred technique preserves the highest spatial resolution for each color component while allowing the parameter instrument to select colors which enhances the clinical value of the resulting image. A suitable color imaging module 16 is the Mightex Systems 5 megapixel monochrome CMOS board level array, used in conjunction with sequential red, green, and blue illumination. The color imaging module preferably includes polarization filtering, which removes interfering specular highlights in reflections from wet or glossy tissue, which is common in injured skin, thereby improving the resulting image quality.

The second imaging technique is rapid non-contact surface mapping, used to capture and accurately measure dimensional data on the imaging subject. Various versions of surface mapping exist as commercial products and are either laser-based or structured light scanners, or stereophotogrammetry. Surface mapping has been applied in medicine to measure wound progression, body surface area, scar changes and cranio-facial asymmetry as well as to create orthodontic and other medically-related devices. The availability of three-dimensional data of body surfaces like the face is becoming increasingly important in many medical specialties such as anthropometry, plastic and maxillo-facial surgery, neurosurgery, visceral surgery, and forensics. When used in medicine, surface images assist medical professionals in diagnosis, analysis, treatment monitoring, simulation, and outcome evaluation. Surface mapping is also used for custom orthotic and prosthetic device fabrication. 3D surface data can be registered and fused with 3D CT, MRI, and other medical imaging techniques to provide a comprehensive view of the patient from the outside in.

Examples of the application of surface mapping include the ability to better understand the facial changes in a developing child and to determine if orthodontics influences facial growth. Surface maps from children scanned over time were compared, generating data as absolute mean shell deviations, standard deviations of the errors during shell overlaps, maximum and minimum range maps, histogram plots, and color maps. Growth rates for male and female children were determined, mapped specifically to facial features in order to provide normative data. Another opportunity is the use of body surface mapping as a new alternative for breast volume computation. Quantification of the complex breast region can be helpful in breast surgery, which is shaped by subjective influences. However, there is no generally recognized method for breast volume calculation. Volume calculations from 3D surface scanning have demonstrated a correlation with volumes measured by MRI (r=0.99). Surface mapping is less expensive and faster than MRI, producing the same results. Surface mapping has also been used to quantitatively assess wound-healing rates. As another example, non-contact color surface maps may be used for segmentation and quantification of hypertrophic scarring resulting from burns. The surface data in concert with digital color images presents new insight into the progression and impact of hypertrophic scars.

Included in the present invention is a surface mapping module 18. Preferably, the surface mapping module 18 offers high spatial resolution and real time operation, is small and lightweight, and has comparatively low power consumption. In one embodiment, the surface mapping module 18 includes an imaging array and a structured light pattern projector 20 spaced apart from the imaging array. In one embodiment, the surface mapping module 18 may be based upon the surface mapping technology developed by Artec Group, Inc., whereby the structured light pattern projector 20 projects a structured pattern of light onto the imaging subject, which is received by the imaging array. Curvature in the imaging subject causes distortions in the received structured light pattern, which may be translated into a three dimensional surface map by appropriate software, as is known in the art. The surface mapping module 18 is capable of imaging surfaces in motion, eliminating any need to stabilize or immobilize an individual or body part of an individual being scanned.

The third imaging technique is digital infrared thermal imaging (“DITI”). DITI is a non-invasive clinical imaging procedure for detecting and monitoring a number of diseases and physical injuries by showing the thermal abnormalities present in the body. It is used as an aid for diagnosis and prognosis, as well as monitoring therapy progress, within many clinical fields, including early breast disease detection, diabetes, arthritis, soft tissue injuries, fibromyalgia, skin cancer, digestive disorders, whiplash, and inflammatory pain. DITI graphically presents soft tissue injury and nerve root involvement, visualizing and recording “pain.” Arthritic disorders generally appear “hot” compared to unaffected areas. Simply recording differences in contralateral regions identifies areas of concern, disease, or injury.

Included in the present invention is a thermal imaging module 22. Preferably, the thermal imaging module 22 is small and lightweight, uncooled, and has low power requirements. In one embodiment, the thermal imaging module 22 is a microbolometer array. Preferably, the microbolometer array has a sensitivity of 0.1° C. or better. A suitable microbolometer array is a thermal imaging core offered by L-3 Communications Infrared Products.

The fourth imaging technique is perfusion imaging, used to directly measure microcirculatory flow. Commercial laser Doppler scanners, one means of perfusion imaging, have been used in clinical applications that include determining burn injury, rheumatoid arthritis, and the health of post-operative flaps. During the inflammatory response to burn injury, there is an increase in perfusion. Laser Doppler imaging (“LDI”), used to assess perfusion, can distinguish between superficial burns, areas of high perfusion, and deep burns, areas of very low perfusion. Laser Doppler perfusion imaging has also been finding increasing utility in dermatology. LDI has been used to study allergic and irritant contact reactions, to quantify the vasoconstrictive effects of corticosteroids, and to objectively evaluate the severity of psoriasis by measuring the blood flow in psoriatic plaques. It has also been used to study the blood flow in pigmented skin lesions and basal cell carcinoma where it has demonstrated significant variations in the mean perfusion of each type of lesion, offering a noninvasive differential diagnosis of skin tumors.

When a diffuse surface such as human skin is illuminated with coherent laser light, a random light interference effect known as a speckle pattern is produced in the image of that surface. If there is movement in the surface, such as capillary blood flow within the skin, the speckles fluctuate in intensity. These fluctuations can be used to provide information about the movement. LDI techniques for blood flow measurements are based on this basic phenomenon. While LDI is becoming a standard, it is limited by specular artifacts, low resolution, and long measurement times.

Included in the present invention is a perfusion imaging module 24. In one embodiment, the perfusion imaging module 24 is a laser Doppler scanner. In this embodiment, the perfusion imaging module includes a coherent light source 26 to illuminate a surface and at least one imaging array to detect the resulting speckle pattern. In a preferred embodiment, the perfusion imaging module 24 includes a plurality of imaging arrays, each receiving identical spectral content, which sequentially acquire temporally offset images. The differences between these temporally offset images can be analyzed to detect time-dependent speckle fluctuation. A preferred technique for perfusion imaging is described in a co-pending U.S. patent application for a “System and Method for Real-Time Perfusion Imaging” filed by the inventors and incorporated herein by reference.

The fifth imaging technique is Near Infrared Spectroscopy (“NIRS”). Skin moisture is a measure of skin health, and can be measured using non-contact NIRS. The level of hydration is one of the significant parameters of healthy skin. The ability to image the level of hydration in skin would provide clinicians a quick insight into the condition of the underlying tissue.

Water has a characteristic optical absorption spectrum. In particular, it includes a distinct absorption band centered at about 1460 nm. Skin hydration can be detected by acquiring a first “data” image of an imaging subject at a wavelength between about 1380-1520 nm, preferably about 1460 nm, and a second “reference” image of an imaging subject at a wavelength less than the 1460 nm absorption band, preferably between about 1100-1300 nm. The first and second images are acquired using an imaging array, such as a NIR sensitive CMOS imaging array. The first and second images are each normalized against stored calibration images of uniform targets taken at corresponding wavelengths. A processor performs a pixel by pixel differencing, either by subtraction or ratio, between the normalized first image and the normalized second image to create a new hydration image. False coloring is added to the hydration image based on the value at each pixel. The hydration image is then displayed to the user on a display. By performing these steps multiple times per second, the user can view skin hydration in real-time or near real-time.

Included in the present invention is a NIRS module 28. In one embodiment, this module 28 includes an imaging array with NIR sensitivity, and an integrated light source 30 or light filtering means capable of providing near infrared light to the imaging array.

Each of the five imaging techniques produce measurements, numerical values which describe skin parameters such as color, contour, temperature, microcirculatory flow, and hydration. Quantitative determination of these parameters allows quantitative assessment skin maladies, such as, for example, burns, erythema, or skin discoloration, which are normally evaluated only by eye and experience. Each of the imaging techniques in the convergent parameter instrument may be used separately, but additional information may be revealed when images acquired by different techniques are integrated to provide combined images.

Each of the five imaging modules preferably includes a signal transmitting unit, a processor which converts raw data into image files, such as bitmap files. This pre-processing step allows each imaging module to provide the same format of data to the central processing unit (“CPU”) 32, a processor, of the convergent parameter instrument, which reduces the workload of the CPU 32 and simplifies integration of images. The CPU 32 serves to process images, namely, analyzing, quantifying, and manipulating image data acquired by the imaging modules or transferred to the instrument 10.

The surface mapping module 18, NIRS module 28, perfusion imaging module 24, and color imaging module 16 each utilize imaging arrays, such as CMOS arrays. In a preferred embodiment, a given imaging array may be used by more than one module by controlling the illumination of the imaging subject. For example, an imaging array may be used to acquire an image as part of the color imaging module 16 by sequentially illuminating the imaging subject with red, green, and blue light. The same imaging array may later be used to acquire an image as part of the NIRS module 28 by illuminating the imaging subject with light at NIR wavelengths. In this preferred embodiment, fewer imaging arrays would be needed, decreasing the cost of the convergent parameter instrument 10.

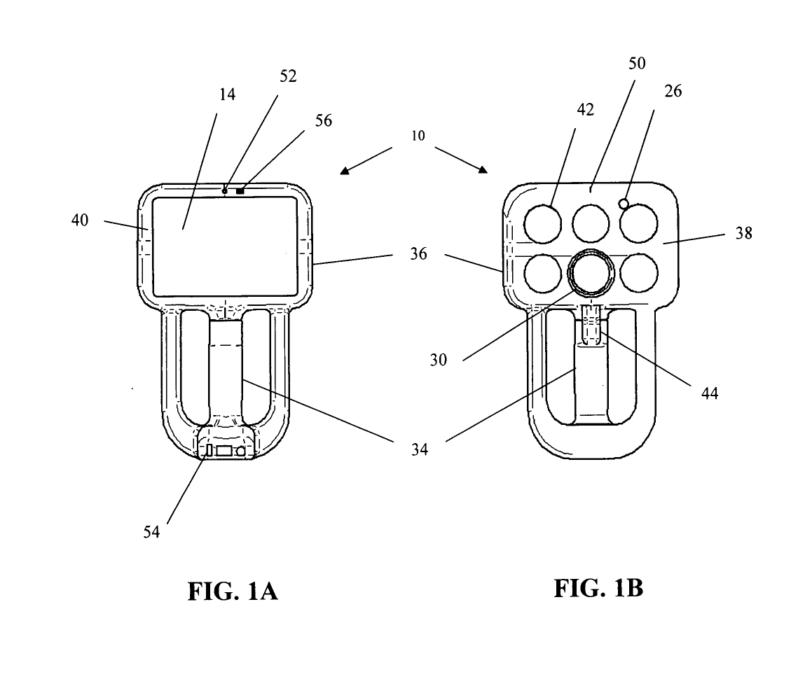

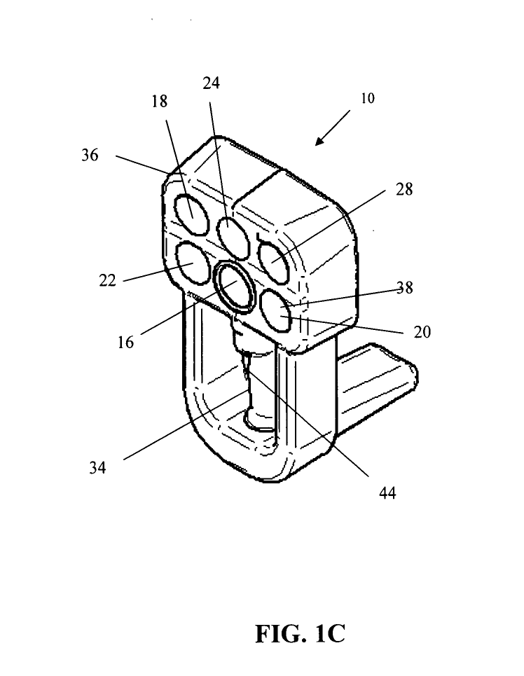

FIGS. 1A, 1B, and 1C depict an embodiment of the present invention. The convergent parameter instrument 10 is shown comprising a handle 34 attached to a body 36. The body 36 includes a first side 38 and a second side 40. The first side 38 includes one or more apertures 42. In this embodiment, each of the one or more apertures 42 is associated with a single imaging module located within the body 36 and allows electromagnetic radiation to reach the imaging module. In a preferred embodiment, the instrument 10 includes six apertures 42, each associated with one of the five imaging modules described herein (the surface mapping module 18 uses two apertures 42, one for the imaging array and one for the structured light pattern projector 20). In alternate embodiments, the instrument 10 may include a single aperture 42 associated with multiple imaging modules or any other suitable combination of apertures and modules. For example, in an embodiment where the same imaging array is used with multiple modules, the instrument 10 may include three apertures 42; one for the thermal imaging module 22, one of the structured light pattern projector 20, and one for the imaging arrays which collect color, surface maps, kin hydration, and perfusion data.

The present invention includes a common display 14, whereby images acquired by each imaging technique are displayed on the same display 14.

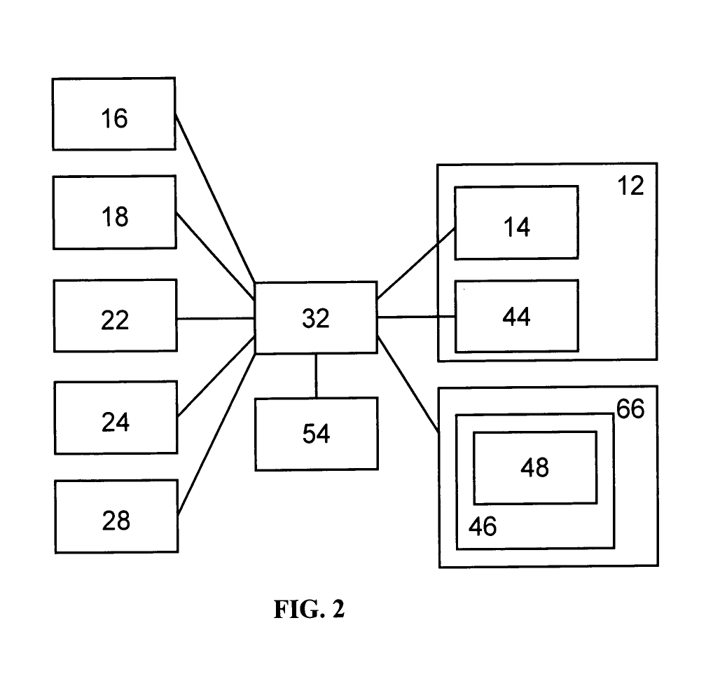

The present invention includes a common control set 12 (FIG. 2) which controls all imaging modalities and functions of the present invention. In a preferred embodiment, the common control set 12 includes the display 14, the display 14 being a touch screen display capable of receiving user input, and an actuator 44. In the embodiment displayed in FIGS. 1A, 1B, and 1C, the actuator 44 is a trigger. In other embodiments, the actuator 44 may be a button, switch, toggle, or other control. In the displayed embodiment, the actuator 44 is positioned to be operable by the user while the user holds the handle 34.

The actuator 44 initiates image acquisition for an imaging module. The touch screen display 14 is used to control which imaging module or modules are activated by the actuator 44 and the data gathering parameters for that module or modules. The actuator 44 effectuates image acquisition for all imaging modules, simplifying the use of the instrument 10 for the user. For example, the user may simply select a first imaging technique using the touch screen display 14, and squeeze the actuator 44 to acquire an image using the first imaging module. Alternatively, the user may select first, second, third, fourth, and fifth imaging techniques using the touch screen display 14, and squeeze the actuator 44 a single time to sequentially acquire images using the five modules. The instrument 10 may also provide a real-time or near real-time “current view” of a given imaging module to the user. In one embodiment, this current view is activated by partially depressing the trigger actuator 44. The instrument 10 continuously displays images from a given module, updating the image presented on the display 14 multiple times per second. Preferably, newly acquired images are be displayed 30-60 times per second, and ideally at a frame rate of about 60 times per second, to provide a latency-free viewing experience to the user. Using this functionality, the user may “preview” images on the display 14 before acquiring and recording the images using a full depression of the trigger actuator 44. In this embodiment, the actuator 44 is a dual function actuator 44, effectuating both preview and acquisition of images.

In a preferred embodiment, the instrument 10 is supportable and operable by a single hand of the user. For example, in the embodiment shown in FIGS. 1A, 1B, and 1C, the user's index finger may control the trigger actuator 44 and the user's remaining fingers and thumb grip the handle 34 to support the instrument 10. The user may use his or her other hand to manipulate the touch screen display 14 then, once imaging modules have been selected, preview and acquire images while controlling the instrument with a single hand.

The instrument 10 includes an electronic system for image analysis 46, namely, software integrated into the instrument 10 and run by the CPU 32 which provides the ability to overlay, combine, and integrate images generated by different imaging techniques or imported into the instrument 10. Texture mapping is an established technique to map 2D images (such as the high resolution color images, thermal images, perfusion images, and NIR images) onto the surface of the 3D model acquired using the surface mapping module 18. This technique allows a user to interact with several forms of data simultaneously. This electronic system for image analysis 46 allows users to acquire, manipulate, register, process, visualize, and manage image data on the handheld instrument 10. Software programs to acquire, manipulate, register, process, visualize, and manage image data are known in the art.

In a preferred embodiment, the electronic system for image analysis 46 includes a database of reference images 48. For example, a user of the instrument 10 may compare an acquired image and a reference image using a split screen view on the display 14. The reference image may be a previously acquired image from the same imaging subject, such that the user may evaluate changes in the imaging subject's skin condition over time. The reference image may also be an exemplary image of a particular feature, such as a particular type of skin cancer or severity of burn, such that a user can compare an acquired image of a similar feature on an imaging subject with the reference image to aid in diagnosis. In one embodiment, the user may insert acquired images into the database of reference images 48 for later use.

In one embodiment, the system for image analysis may include a patient positioning system (“PPS”) to aid the comparison of acquired images to a reference image. The user may use the touch screen display 14 to select the PPS prior to acquiring images of the imaging subject. Upon selection of PPS, the user browses through the database of reference images 48 and selects a desired reference image. The display 14 then displays both the selected reference image and the current view of the instrument 10, either in a split screen view or by cycling between the reference image and current view. The user may then position the instrument 10 in relation to the imaging subject to align the current view and reference image. When the user acquires images of the imaging subject, they will be at the same orientation as the reference image, simplifying comparison of the acquired images and the reference image. In one embodiment, the instrument 10 may include image matching software to assist the user in aligning the current view of the imaging subject and the reference image.

The electronic system for image analysis 46 is accessed through the touch screen display 14 and is designed to maximize the value of the portability of the present invention. Other methods of image analysis include acquiring two images of the same body feature at different dates and comparing the changes in the body feature. Images may be acquired based on a plurality of imaging techniques, the images integrated into a combined image or otherwise manipulated, and reference images provided all on the handheld instrument 10, offering unprecedented mobility in connection with improvements to the accuracy and speed of evaluation of skin maladies. Due to the self-contained, handheld nature of the instrument 10, it is particularly suited to being used to evaluate skin maladies, such as burns, at locations remote from medical facilities. For example, an emergency medical technician could use the instrument 10 to evaluate the severity of a burn at the location of a fire, before the burn victim is taken to a hospital.

The instrument 10 includes light sources according to the requirements of each imaging technique. The instrument 10 includes an integrated, spectrally chosen, stable light source 30, such as a ring of solid state lighting, which includes polarization filtering. In one embodiment, the integrated light source 30 is preferably a circular array of discrete LEDs. This array includes LEDs emitting wavelengths appropriate for color images as well as LEDs emitting wavelengths in the near infrared. Each LED preferably includes a polarization filter appropriate for its wavelength. In another embodiment, the integrated light source 30 may be two separate circular arrays of discrete LEDs, one with LEDs emitting wavelengths appropriate for color imaging and the other with LEDs emitting wavelengths appropriate for NIR imaging. The integrated light source 30, whether embodied in one or two arrays of LEDs, preferably emits in wavelengths ranging from about 400 nm to about 1600 nm. The surface mapping module includes a structured light pattern projector 20 as the light source. Preferably, the structured light pattern projector 20 of the surface mapping module 18 is located at the opposite corner of the body 36 from the imaging array of the surface mapping module 18 to provide the needed base separation required for accurate 3D profiling. A coherent light source 26 is included for the perfusion imaging module 24. Preferably, the coherent light source 26 is a 10 mW laser emitting between about 630-850 nm to illuminate a field of view of about six inches diameter at a distance of about three feet. Thermal imaging requires no additional light source as infrared radiation is provided by the imaging subject. The imaging optics for all imaging modules are designed to provide a similar field of view focused at a common focal distance.

The common field of view and focal distance of the present invention simplifies image registration and enhances the accuracy of integrated images. In one embodiment, the common focal distance is about three feet. In a preferred embodiment, the instrument 10 includes an integrated range sensor 50 and a focus indicator 52 in electronic communication with the range sensor 50. The range sensor 50 is located on the first side 38 of the instrument 10 and the focus indicator 52 is located on the second side 40 of the instrument 10. The range sensor 50 and focus indicator 52 cooperatively determine the range to the imaging subject and signal to the user whether the imaging subject is located at the common focal distance. A suitable range sensor 50 is the Sharp GP2Y0A02YK IR Sensor. In one embodiment, the focus indicator 52 is a red/green/blue LED which emits red when the range sensor 50 detects that the imaging subject is too close, green when the imaging subject is in focus, and blue when the imaging subject is too far.

In a preferred embodiment, the instrument 10 includes data transfer unit 54 for transferring electronic data to and from the instrument 10. The data transfer unit 54 may be used to transfer image data to and from the instrument 10, or introduce software updates or additions to the database of reference images 48. The data transfer unit 54 may be at least one of a USB port, integrated wireless network adapter, Ethernet port, IEEE 1394 interface, serial port, smart card port, or other suitable means for transferring electronic data to and from the instrument 10.

In a preferred embodiment, the instrument 10 includes an integrated audio recording and reproduction unit 56, such as a combination microphone/speaker. This feature allows the user to record comments to accompany acquired images. This feature may also be used to emit audible cues for the user or replay recorded sounds. In one embodiment, the audio recording and reproduction unit 56 emits an audible cue to the user when data acquisition is complete, indicating that the actuator 44 may be released.

The instrument 10 depicted in FIGS. 1A, 1B, and 1C is only one embodiment of the present invention. Alternative constructions of the instrument 10 are contemplated which lack a handle 34. In such alternative constructions, the actuator 44 may be located on the body 36 or may be absent and all functions controlled by the touch screen display 14. In other embodiments, the display 14 may not be a touch screen display and may simply serve as an output device. In such embodiments, the common control set 12 would include at least one additional input device, such as, for example, a keyboard. In all embodiments, the instrument 10 is most preferably portable and handheld.

Referring now to FIG. 2, the present invention includes a CPU 32 in electronic communication with a color imaging module 16, surface mapping module 18, thermal imaging module 22, perfusion imaging module 24, and NIRS imaging module 28. The CPU 32 is also in electronic communication with a common control set 12, computer readable storage media 58, and may receive or convey data via a data transfer unit 54. The common control set 12 comprises the display 14, in its role as a touch screen input device, and actuator 44. The computer readable storage media 58 stores images acquired by the instrument 10, the electronic system for image analysis 46, and image data transferred to the instrument 10.

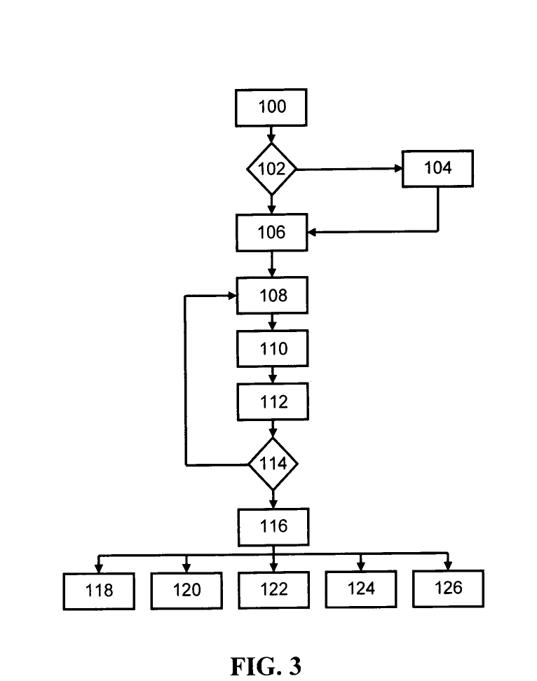

FIG. 3 depicts a method of using a convergent parameter instrument 10. In step 100, a user selects an imaging subject. In step 102, the user chooses whether to use the PPS. If so, the user selects a reference image from the database of reference images 48 in step 104. In step 106, the user uses the common display 14 to select at least one imaging technique to determine a skin parameter. In step 108, the user orients the instrument 10 in the direction of the imaging subject. In step 110, the user adjusts the distance between the instrument 10 and the imaging subject to place the imaging subject in focus, as indicated by the focus indicator 52. In step 112, where the actuator 44 is a trigger, the user partially depresses the actuator 44 to view the current images of the selected modules on the display 14. The images are presented sequentially at a user programmable rate. In step 114, the user determines whether the current images are acceptable. If the user elected to use the PPS in step 102, the user also determines the acceptability of the current images by evaluating whether the current images are aligned with the selected reference image. If the current images are unacceptable, the user returns to step 108. Otherwise, the user fully depresses the actuator 44 to acquire the current images in step 116. Once images are acquired, the user may elect to further interact with the images by proceeding with at least one processing and analysis step. In step 118, the user compares the acquired images to previously acquired images or images in the database of reference images 48. In step 120, the user adds audio commentary to at least one of the acquired images using the audio recording and reproduction unit 56. In step 122, the user stitches, crops, annotates, or otherwise modifies at least one acquired image. In step 124, the user integrates at least two acquired images into a single combined image. In step 126, the user downloads at least one acquired image to removable media or directly to a host computer via the data transfer unit 54.

For an example of the use of the convergent parameter instrument 10, a clinician may wish to document the state of a pressure ulcer on the bottom of a patient's foot and is interested in the skin parameters of color, contour, perfusion, and temperature. The clinician does not desire to use the PPS. Using the touch screen display 14, the clinician selects the color imaging module 16, the surface mapping module 18, the perfusion imaging module 24, and the thermal imaging module 22. The clinician then aims the instrument 10 at the patient's foot, confirms the range is acceptable using the focus indicator 52, and partially depresses the actuator 44. The display 14 then sequentially presents the current views of each selected imaging module in real time. The clinician adjusts the position of the instrument 10 until the most desired view is achieved. The clinician then fully depresses the actuator 44 to acquire the images. Acquisition may require up to several seconds depending on the number of imaging modules selected. Acquired images are stored in computer readable storage media 58, from which they may be reviewed and processed. Processing may occur immediately using the instrument 10 itself or later at a host computer.

In a preferred embodiment, acquired images are stored using the medical imaging standard DICOM format. This format is used with MRI and CT images and allows the user to merge or overlay images acquired using the instrument 10 with images acquired using MRI or CT scans. Images acquired using MRI or CT scans may be input into the instrument 10 for processing using the electronic system for image analysis of the instrument 10. Alternatively, images acquired using the instrument 10 may be output to a host computer and there combined with MRI or CT images.

Although the present invention is discussed in terms of diagnosis, evaluation, monitoring, and treatment of skin disorders and damage, the present invention may be used in connection with medical conditions apart from skin or for non-medical purposes. For example, the present invention may be used in connection with the development and sale of cosmetics, as a customer's skin condition can be quantified and an appropriate cosmetic offered. The present invention may also be used by a skin chemist developing topical creams or other health or beauty aids, as it would allow quantified determination of the efficacy of the products.

The convergent parameter instrument 10 of the present invention is modular in nature. The inventors anticipate future improvements in imaging technology for quantifying the five skin parameters. The present invention is designed such that, for example, a NIRS module 28 based on current technology could be replaced with an appropriately shaped NIRS module 28 of similar or smaller size based on more advanced technology. Each module is in communication with the CPU 32 using a standard electronic communication method, such as a USB connection, such that new modules of the appropriate size and shape may be simply plugged in. Such replacements may require a user to return his or her convergent parameter instrument 10 to the manufacturer for upgrades, although the inventors contemplate adding new modules in the field in future embodiments of the invention. New software can be added to the instrument 10 using the data transfer unit 54 to allow the instrument 10 to recognize and control new or upgraded modules.

When used for certain purposes, not all five imaging modules may be necessary to perform the functions desired by the user. In one embodiment, the instrument 10 may include less than five imaging modules, such as a least one imaging module, at least two imaging modules, at least three imaging modules, or at least four imaging modules. Any combination of imaging modules may be included, based on the needs of the user. A user may purchase an embodiment of the present invention including less than all five of the described imaging modules, and have at least one additional module incorporated into the body 36 of the instrument 10 at a later time. The modular design of the instrument 10 allows for additional modules to be controllable by the common control set 12 and images acquired using the additional modules to viewable on the common display 14.

The foregoing detailed description is given primarily for clearness of understanding and no unnecessary limitations are to be understood therefrom for modifications can be made by those skilled in the art upon reading this disclosure and may be made without departing from the spirit of the invention and scope of the appended claims.

What is claimed is:

1. A convergent parameter instrument comprising:
at least two imaging modules, each of the at least two imaging modules being selected from the group consisting of: a color imaging module, a surface mapping module, a thermal imaging module, a perfusion imaging module, and a near infrared spectroscopy module;
a common control set for controlling each of the at least two imaging modules;
a common display for displaying images acquired by each of the at least two imaging modules; and
a central processing unit for processing images acquired by each of the at least two imaging modules, the central processing unit in electronic communication with each of the at least two imaging modules, the common control set, and the common display.

2. The convergent parameter instrument of claim 1, further comprising a light source controllable by the common control set.

3. The convergent parameter instrument of claim 2, wherein said light source is capable of emitting at wavelengths between about 400 nm-1600 nm.

4. The convergent parameter instrument of claim 2, wherein said light source is a coherent light source capable of emitting at a wavelength between about 630-850 nm.

5. The convergent parameter instrument of claim 2, wherein said light source is a structured light projector.

6. The convergent parameter instrument of claim 2, wherein said light source comprises an array of light emitting diodes emitting at wavelengths between about 400 nm-1600 nm, a coherent light source capable of emitting at a wavelength between about 630-850 nm, and a structured light projector.

7. The convergent parameter instrument of claim 1, further comprising an integrated range sensor.

8. The convergent parameter instrument of claim 1, further comprising an integrated audio recording and reproduction unit.

9. The convergent parameter instrument of claim 1, further comprising computer readable storage media in electronic communication with said central processing unit.

10. The convergent parameter instrument of claim 1, further comprising a data transfer unit in electronic communication with said central processing unit.

11. The convergent parameter instrument of claim 1, wherein said common display is said touch screen display.

12. The convergent parameter instrument of claim 1, wherein said instrument further comprises an electronic system for image analysis.

13. The convergent parameter instrument of claim 12, wherein said system for image analysis includes a database of reference images.

14. The convergent parameter instrument of claim 1, wherein said color imaging module comprises a high resolution imaging array and means for providing visible light to the high resolution imaging array.

15. The convergent parameter instrument of claim 1, wherein said surface mapping module comprises an imaging array and a structured light projector spaced apart from the imaging array.

16. The convergent parameter instrument of claim 1, wherein said thermal imaging module comprises a microbolometer array.

17. The convergent parameter instrument of claim 1, wherein said perfusion imaging module comprises a coherent light source and a plurality of imaging arrays which each acquire temporally offset images.

18. The convergent parameter instrument of claim 1, wherein said near infrared spectroscopy module comprises a near infrared sensitive imaging array and means for providing near infrared light to said near infrared sensitive imaging array.

19. The convergent parameter instrument of claim 1, wherein said instrument is handheld.

20. The convergent parameter instrument of claim 1, wherein each of said modules share a common field of view and focal length.

21. The convergent parameter instrument of claim 1, wherein said at least two imaging modules are at least three imaging modules.

22. The convergent parameter instrument of claim 1, wherein said at least two imaging modules are at least four imaging modules.

23. The convergent parameter instrument of claim 1, wherein said at least two imaging modules are five imaging modules.

24. The convergent parameter instrument of claim 1, further comprising an imaging array used by at least two of said at least two imaging modules to acquire image data.

25. A convergent parameter instrument comprising a body incorporating a common display, a common control set, a central processing unit, and between two and four imaging modules selected from the group consisting of:
a color imaging module;
a surface mapping module;
a thermal imaging module;
a perfusion imaging module; and
a near infrared spectroscopy module;
wherein said central processing unit is in electronic communication with said common display, said common control set, and each of said selected imaging modules;
wherein each of said selected imaging modules are controllable using said common control set;
wherein images acquired by each of said selected imaging modules are viewable on said common display; and
wherein said instrument is capable of incorporating at least one additional module from said group into said body, said at least one additional module, once incorporated, being controllable using said common control set and in electronic communication with said central processing unit, and wherein images acquired by said at least one additional module are viewable on said common display.

26. A method for quantitatively assessing an imaging subject's skin, comprising:
a. acquiring at least two skin parameters using a convergent parameter instrument through a combination of at least two imaging techniques, each of the at least two imaging techniques being selected from the group consisting of:
1. acquiring high resolution color image data using a high resolution color imaging module;
2. acquiring surface mapping data using a surface mapping module;
3. acquiring thermal image data using a thermal imaging module;
4. acquiring perfusion image data using a perfusion imaging module; and
5. acquiring hydration data using a near infrared spectroscopy module;
b. using said convergent parameter instrument to select and quantify an imaging subject feature visible in said at least one image; and
c. assessing said imaging subject based on said quantified imaging subject feature.

27. The process of claim 26, wherein said convergent parameter instrument includes a database of reference images and wherein step c further comprises assessing said imaging subject's skin based on a comparison of said imaging subject's skin to a reference image.

28. The process of claim 27, wherein said reference image is an earlier acquired image of the same imaging subject.

29. The process of claim 27, wherein said reference image is an exemplary image of a particular skin feature.

30. The process of claim 27, wherein the step of acquiring at least two skin parameters in step a further includes aligning the acquired images with the reference image.

31. The process of claim 26, wherein said imaging subject feature is quantified in step b by using said convergent parameter instrument to determine at least one numerical value describing said imaging subject feature.

32. The process of claim 26, wherein:
at least two images are acquired in step a using different imaging techniques;
step a further comprises using said convergent parameter instrument to integrate said at least two images into a combined image;
said imaging subject feature in step b is visible in said combined image.

33. A method for evaluating a skin malady at a location remote from medical facilities comprising the steps of:
a. selecting a reference image of the type of malady to be evaluated from a database of reference images;
b. acquiring at least one image of the skin malady using a handheld convergent parameter instrument;
c. evaluating the skin malady by comparing the at least one acquired image with the reference image;
wherein the database of reference images is accessed using the handheld convergent parameter instrument.
